SEQUENCE LISTING (26 sequences)

SEQ ID NO: 1 - protein, 14 aa, H. sapien
AEAGVPAEFSIWTR

SEQ ID NO: 2 - protein, 16 aa, H. sapien
AFGPGLQGGSAGSPAR

SEQ ID NO: 3 - protein, 28 aa, H. sapien
CAPGVVGPAEADIDFDIIRNDNDTFTVK

SEQ ID NO: 4 - protein, 10 aa, H. sapien
DKGEYTLVVK

SEQ ID NO: 5 - protein, 24 aa, H. sapien
EAGAGGLAIAVEGPSKAEISFEDR

SEQ ID NO: 6 - protein, 11 aa, H. sapien
EATTEFSVDAR

SEQ ID NO: 7 - protein, 14 aa, H. sapien
FIPRENGVYLIDVK

SEQ ID NO: 8 - protein, 12 aa, H. sapien
GAGSGELKVTVK

SEQ ID NO: 9 - protein, 18 aa, H. sapien
GAGTGGLGLAVEGPSEAK

SEQ ID NO: 10 - protein, 25 aa, H. sapien
GAGTGGLGLAVEGPSEAKMSCMDNK

SEQ ID NO: 11 - protein, 5 aa, H. sapien
GLQPK

SEQ ID NO: 12 - protein, 10 aa, H. sapien
GTVEPQLEAR

SEQ ID NO: 13 - protein, 13 aa, H. sapien
IANLQTDLSDGLR

SEQ ID NO: 14 - protein, 8 aa, H. sapien
LLGWIQNK

SEQ ID NO: 15 - protein, 11 aa, H. sapien
LPQLPITNFSR

SEQ ID NO: 16 - protein, 9 aa, H. sapien
LSPFMADIR

SEQ ID NO: 17 - protein, 15 aa, H. sapien
SPFSVAVSPSLDLSK

SEQ ID NO: 18 - protein, 16 aa, H. sapien
TGVAVNKPAEFTVDAK

SEQ ID NO: 19 - protein, 16 aa, H. sapien
TGVELGKPTHFTVNAK

SEQ ID NO: 20 - protein, 9 aa, H. sapien
TPCEEILVK

SEQ ID NO: 21 - protein, 16 aa, H. sapien
VANPSGNLTETYVQDR

SEQ ID NO: 22 - protein, 17 aa, H. sapien
VAQPTITDNKDGTVTVR

SEQ ID NO: 23 - protein, 17 aa, H. sapien
VTAQGPGLEPSGNIANK

SEQ ID NO: 24 - protein, 12 aa, H. sapien
VTVLFAGQHIAK

SEQ ID NO: 25 - protein, 12 aa, H. sapien
YGGDEIPFSPYR

SEQ ID NO: 26 - protein, 12 aa, H. sapien
YGGQPVPNFPSK

(19) United States
(12) Patent Application Publication
(10) Pub. No.: US 2006/0063214 A1
(43) Pub. Date: Mar. 23, 2006

(54) Methods and compositions for diagnosing neoplastic disease

(76) Inventor: Ozge Alper, Bethesda, MD (US)

Correspondence Address: TODD B. BUCK, Ph.d.;CASTELLANO MALM FERRARIO &bUCK LLC, 8500 LEESBURG PIKE, SUITE 402, VIENNA,, VA, 22182, US

(21) Appl. No.: 10/943,793

(22) Filed: Sep. 17, 2004

(51) Int. Cl. G01N 33/574

(52) U.S. Cl. 435/7.23

(57) ABSTRACT

Methods and compositions for determining whether a subject at least has a neoplastic disease are provided. In practicing the subject methods, a sample from a subject is assayed for a soluble filamin analyte, such as a filamin A analyte, to determine whether the subject at least has the neoplastic disease. Also provided are kits, systems, and devices for practicing the subject methods.

### BACKGROUND OF THE INVENTION

In spite of numerous advances in medical research, cancer remains the second leading cause of death in the United States. Traditional modes of clinical care, such as surgical resection, radiotherapy and chemotherapy, have a significant failure rate, especially for solid tumors. Failure occurs either because the initial tumor is unresponsive, or because of recurrence due to regrowth at the original site and/or metastases. Even in cancers such as breast cancer where the mortality rate has decreased, successful intervention relies on early detection of the cancerous cells. The etiology, diagnosis and ablation of cancer remain a central focus for medical research and development.

Current methods of cancer treatment are relatively non-selective. Surgery removes the diseased tissue, radiotherapy shrinks solid tumors and chemotherapy kills rapidly dividing cells. Chemotherapy, in particular, results in numerous side effects, in some cases so severe to preclude the use of potentially effective drugs. Moreover, cancers often develop resistance to chemotherapeutic drugs.

Since the chance for complete remission of cancer is, in most cases, greatly enhanced by early diagnosis, it is desirable that physicians be able to detect cancers before a substantial tumor develops. However, the development of methods that permit rapid and accurate detection of many forms of cancers continues to challenge the medical community. Two such illustrative forms of cancer are breast cancer and ovarian cancer. Thus a major problem in the treatment of cancer remains its early detection, which enables therapeutic treatment from the onset of the disease resulting in successful treatment in many cases.

Accordingly, there remains a need in this art for developing methods for early detection of neoplastic disease in order to make clinical decisions on therapy, disease progression surveillance, responsiveness to therapy (e.g., staging of the disease), the likelihood of disease progression, and the like. The present invention addresses this need.

Relevant Literature

Gorlin, et al., 1990, J. Cell Biol. 111(3):1089-1105; Vadlamudi et al., 2002, Nat Cell Biol. 4(9):681-90; Flanagan et al., 2001, J. Cell Biol. 155(4):511-7; Alper et al., 1990 Cell Growth Differ. 1 (12):591-9; Xie et al., 1998, Biochem. Biophys. Res. Commun. 251 (3), 914-919); Takafuta et al., 1998, J. Biol. Chem. 273 (28), 17531-17538.

### SUMMARY OF THE INVENTION

Methods and compositions for determining whether a subject at least has a neoplastic disease are provided. In practicing the subject methods, a sample from a subject is assayed for a soluble filamin analyte, such as a filamin A analyte, to determine whether the subject at least has the neoplastic disease. Also provided are kits, systems, and devices for practicing the subject methods.

One feature of the invention provides a method of diagnosing whether a subject has a neoplastic disease, including assaying an extracellular sample from a subject for a soluble filamin analyte to diagnose whether the subject has a neoplastic disease. In some embodiments the soluble filamin analyte is a soluble filamin A analyte. In some embodiments, the extracellular sample is a blood sample. In certain embodiments the extracellular sample is qualititatively assayed for the soluble filamin analyte. In other embodiments the extracellular sample is quantitatively assayed for said soluble filiman analyte.

In some embodiments the assaying employs a filamin analyte affinity reagent, such as an antibody reagent. In certain embodiments, the assaying employs two distinct filamin analyte affinity reagents. In further embodiments, the method is a method of monitoring progression of said neoplastic disease of said subject. In some embodiments the neoplastic disease is a carcinoma, such as breast cancer or ovarian cancer.

Another feature of the invention provides a method of treating a subject having a neoplastic disease, including diagnosing whether a subject suffers from a neoplastic disease by assaying an extracellular sample from the subject for a soluble filamin analyte; and treating the subject based on results from the diagnosing step. In some embodiments, the soluble filamin analyte is a soluble filamin A analyte.

Yet another feature of the invention provides a kit for use in diagnosing whether a subject has a neoplastic disease, including reagents for assaying a sample for a soluble filamin analyte; and a reference. In some embodiments, the soluble filamin analyte is a soluble filamin A analyte. In some embodiments, the reagents include at least one filamin analyte specific affinity reagent, such as an antibody reagent. In further embodiments the affinity reagent includes a detectable label. In certain embodiments, the at least one filamin analyte specific affinity reagent is immobilized on a surface of a solid support. In other embodiments, the kit further includes a sample obtainment element.

Yet another feature of the invention provides a system for use in diagnosing whether a subject has a neoplastic disease, including reagents for assaying a sample for a soluble filamin analyte; and a reference. In some embodiments, the soluble filamin analyte is a soluble filamin A analyte. In some embodiments, the reagents include at least one filamin analyte specific affinity reagent, such as an antibody reagent. In further embodiments the affinity reagent includes a detectable label. In certain embodiments, the at least one filamin analyte specific affinity reagent is immobilized on a surface of a solid support. In other embodiments, the system further includes a sample obtainment element.

Yet another feature of the invention provides a device for use in diagnosing whether a subject suffers from a neoplastic disease, including a soluble filamin analyte specific affinity reagent immobilized on a surface of a solid support. In some embodiments, the soluble filamin analyte is a soluble filamin A analyte. In some embodiments, the soluble filamin analyte specific affinity reagent includes an antibody or binding fragment thereof. In other embodiments, the antibody is a monoclonal antibody.

Yet another feature of the invention provides an antibody that specifically binds to a soluble form of a filamin analyte. In some embodiments, the filamin analyte is a soluble form of a filamin A analyte. In certain embodiments, the antibody is a polyclonal antibody. In other embodiments, the antibody is a monoclonal antibody.

Yet another feature of the invention provides a cell, e.g., a hybridoma, that secretes an antibody that specifically binds to a soluble form of a filamin analyte. In some embodiments, the filamin analyte is a soluble form of a filamin A analyte.

### DEFINITIONS

The terms “neoplastic disease” include premalignant and malignant cellular growth or tumor caused by abnormal and uncontrolled cellular division; wherein the growth may spread to other parts of the body through the lymphatic system or the bloodstream.

The term “carcinoma” means an invasive malignant cellular growth or tumor derived from epithelial tissue of an organ, such as, for example, renal cell carcinoma, squamous cell carcinoma, breast carcinoma, ovarian carcinoma, papillary carcinoma, and the like. The term “carcinoma” includes “carcinoma in situ” which is an early stage of malignant cellular growth in which the tumor is confined to the organ where it first developed. In other words, the disease has not invaded other parts of the organ or spread to distant parts of the body. The term “carcinoma” also includes metastasized forms of the carcinoma, wherein the cancer cells spread to distant locations of the body by way of the lymphatic system or bloodstream.

By “breast carcinoma” is meant an invasive malignant cellular growth or tumor derived from epithelial tissue of the breast and includes ductal carcinoma in situ, invasive ductal carcinoma, lobular carcinoma in situ, invasive lobular carcinoma, medullary carcinoma, and Paget's disease of the nipple.

By “ovarian carcinoma” is meant an invasive malignant cellular growth or tumor derived from epithelial tissue of one or both ovaries.

By “bodily fluid” is meant a naturally occurring fluid of the human body such as serum, plasma, blood, urine, or lymph, particularly blood or blood products and urine.

By “blood sample” is meant a volume of whole blood or fraction thereof, e.g., serum, plasma, etc.

By “disease severity” is meant relative stage of disease progression. Disease severity may be correlated with the impact the disease may have on the patient's overall health or the risk of patient death as a result of disease. The severity of the disease may affect decisions relating to patient treatment subsequent to diagnosis.

The terms “filamin,” “human actin binding protein,” or “human ABP” refers to a family of proteins that crosslink actin filaments into orthogonal networks in cortical cytoplasm and participate in the anchoring of membrane proteins for the actin cytoskeleton. Filamins include three functional domains: an N-termial filamentous actin-binfing domain, a C-terminal self association domain, and a membrane glycoprotein-binding domain. The family of filamin proteins includes the folllowing three proteins: filamin A, filamin B, and filamin C.

The terms “filamin A,” “human filamin A,” “alpha-filamin”, “filamin 1”, “ABP-280” “endothelial actin-binding protein” and “nonmuscle filamin” refer to a 280-kD filamin protein encoded by the FLNA gene, that is a widely expressed protein that regulates reorganization of the actin cytoskeleton by interacting with integrins, transmembrane receptor complexes, and second messengers. The polypeptide sequence of filamin A is available at GenBank Accession No. P21333 (Gorlin, et al., 1990, J. Cell Biol. 111(3):1089-1105).

The terms “filamin B,” “human filamin B,” “beta-filamin”, “ABP-278” “endothelial actin-binding protein” and “nonmuscle filamin” refer to a 278-kD encoded by the FLNB gene that binds actin filaments. The polypeptide sequence of filamin B is available at Gen Bank Accession No. 075369 (Takafuta et al., 1998, J. Biol. Chem. 273 (28), 17531-17538).

The terms “filamin C,” “human filamin C”, “filamin 2”, “gamma filamin”, “ABP-280, Autosomal Form” refer to a 280-kD protein encoded by the FLNC gene that binds actin filaments. The polypeptide sequence of filamin C is available at GenBank Accession No. Q14315 (Xie et al., 1998, Biochem. Biophys. Res. Commun. 251 (3), 914-919).

By “antigenic fragment” of filamin is meant a portion of filamin which is capable of binding an antibody generated by immunization of a mammal with filamin A or a fragment thereof. Preferably, the antibodies which specifically bind an epitope of the isolated antigenic fragment will also bind the same epitope in the context of the native protein from which the fragment was derived.

An “affinity reagent” of the subject invention has an analyte binding domain, moiety or component that has a high binding affinity for a target analyte. By high binding affinity is meant a binding affinity of at least about 10⁻⁴ M, usually at least about 10⁻⁶ M or higher, e.g., 10⁻⁹M or higher. The affinity reagent may be any of a variety of different types of molecules, so long as it exhibits the requisite binding affinity for the target protein when present as tagged affinity ligand.

As such, the affinity reagent may be a small molecule or large molecule ligand. By small molecule ligand is meant a ligand ranging in size from about 50 to about 10,000 daltons, usually from about 50 to about 5,000 daltons and more usually from about 100 to about 1000 daltons. By large molecule is meant a ligand ranging in size from about 10,000 daltons or greater in molecular weight.

The small molecule may be any molecule, as well as binding portion or fragment thereof, that is capable of binding with the requisite affinity to the target protein. Generally, the small molecule is a small organic molecule that is capable of binding to the target analyte of interest. The small molecule will include one or more functional groups necessary for structural interaction with the target analyte, e.g., groups necessary for hydrophobic, hydrophilic, electrostatic or even covalent interactions. Where the target analyte is a protein, the drug moiety will include functional groups necessary for structural interaction with proteins, such as hydrogen bonding, hydrophobic-hydrophobic interactions, electrostatic interactions, etc., and will typically include at least an amine, amide, sulfhydryl, carbonyl, hydroxyl or carboxyl group, preferably at least two of the functional chemical groups. The small molecule may also comprise a region that may be modified and/or participate in covalent linkage to a label component, a substrate surface, or other entity, depending on the particular assay protocol being employed, without substantially adversely affecting the small molecule's ability to bind to its target analyte.

Small molecule affinity ligands often comprise cyclical carbon or heterocyclic structures and/or aromatic or polyaromatic structures substituted with one or more of the above functional groups. Also of interest as small molecules are structures found among biomolecules, including peptides, saccharides, fatty acids, steroids, purines, pyrimidines, derivatives, structural analogs or combinations thereof. Such compounds may be screened to identify those of interest, where a variety of different screening protocols are known in the art.

The small molecule may be derived from a naturally occurring or synthetic compound that may be obtained from a wide variety of sources, including libraries of synthetic or natural compounds. For example, numerous means are available for random and directed synthesis of a wide variety of organic compounds and biomolecules, including the preparation of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known small molecules may be subjected to directed or random chemical modifications, such as acylation, alkylation, esterification, amidification, etc. to produce structural analogs.

As such, the small molecule may be obtained from a library of naturally occurring or synthetic molecules, including a library of compounds produced through combinatorial means, i.e. a compound diversity combinatorial library.

When obtained from such libraries, the small molecule employed will have demonstrated some desirable affinity for the protein target in a convenient binding affinity assay. Combinatorial libraries, as well as methods for the production and screening, are known in the art and described in: U.S. Pat. Nos. 5,741,713; 5,734,018; 5,731,423; 5,721,099; 5,708,153; 5,698,673; 5,688,997; 5,688,696; 5,684,711; 5,641,862; 5,639,603; 5,593,853; 5,574,656; 5,571,698; 5,565,324; 5,549,974; 5,545,568; 5,541,061; 5,525,735; 5,463,564; 5,440,016; 5,438,119; 5,223,409, the disclosures of which are herein incorporated by reference.

As pointed out, the affinity ligand can also be a large molecule. Of particular interest as large molecule affinity ligands are antibodies, as well as binding fragments and mimetics thereof. Where antibodies are the affinity ligand, they may be derived from polyclonal compositions, such that a heterogeneous population of antibodies differing by specificity are each tagged with the same tag nucleic acid, or monoclonal compositions, in which a homogeneous population of identical antibodies that have the same specificity for the target protein are each tagged with the same tag nucleic acid. As such, the affinity ligand may be either a monoclonal or polyclonal antibody. In yet other embodiments, the affinity ligand is an antibody binding fragment or mimetic, where these fragments and mimetics have the requisite binding affinity for the target protein. For example, antibody fragments, such as Fv, F(ab)₂, Fab′ and Fab may be prepared by cleavage of the intact protein, e.g., by protease or chemical cleavage. Also of interest are recombinantly produced antibody fragments, such as single chain antibodies or scFvs, where such recombinantly produced antibody fragments retain the binding characteristics of the above antibodies. Such recombinantly produced antibody fragments generally include at least the VH and VL domains of the subject antibodies, so as to retain the binding characteristics of the subject antibodies. These recombinantly produced antibody fragments or mimetics of the subject invention may be readily prepared using any convenient methodology, such as the methodology disclosed in U.S. Pat. Nos. 5,851,829 and 5,965,371; the disclosures of which are herein incorporated by reference.

The above described antibodies, fragments and mimetics thereof may be obtained from commercial sources and/or prepared using any convenient technology, where methods of producing polyclonal antibodies, monoclonal antibodies, fragments and mimetics thereof, including recombinant derivatives thereof, are known to those of the skill in the art.

Also suitable for use as binding domains are polynucleic acid aptimers.

Polynucleic acid aptamers may be RNA oligonucleotides which may act to selectively bind proteins, much in the same manner as a receptor or antibody (Conrad et al., Methods Enzymol. (1996), 267(Combinatorial Chemistry), 336-367).

By “binds specifically” is meant high avidity and/or high affinity binding of an antibody to a specific antigen. Antibody binding to its epitope on this specific antigen is stronger than binding of the same antibody to any other epitope, particularly those which may be present in molecules in association with, or in the same sample, as the specific antigen of interest. Antibodies which bind specifically to a polypeptide of interest may be capable of binding other polypeptides at a weak, yet detectable, level (e.g., 10% or less of the binding shown to the polypeptide of interest). Such weak binding, or background binding, is readily discernible from the specific antibody binding to the polypeptide of interest, e.g., by use of appropriate controls.

By “detectably labeled affinity reagent”, “detectably labeled antibody”, “detectably labeled filamin” or “detectably labeled filamin fragment” is meant an affinity reagent, e.g., antibody (or antibody fragment which retains binding specificity), filamin, or filamin polypeptide fragment having an attached detectable label. The detectable label may be attached by chemical conjugation, but where the label is a polypeptide, it could alternatively be attached by genetic engineering techniques. Methods for production of detectably labeled proteins are well known in the art. Detectable labels may be selected from a variety of such labels known in the art, but normally are radioisotopes, fluorophores, enzymes (e.g., horseradish peroxidase), or other moieties or compounds which either emit a detectable signal (e.g., radioactivity, fluorescence, color) or emit a detectable signal after exposure of the label to its substrate. Various detectable label/substrate pairs (e.g., horseradish peroxidase/diaminobenzidine, avidin/streptavidin, luciferase/luciferin), methods for labeling antibodies, and methods for using labeled antibodies to detect an antigen (such as fialmin A or filamin A fragments) are well known in the art (see, for example, Harlow and Lane, eds. (Antibodies: A Laboratory Manual (1988) Cold Spring Harbor Laboratory Press, Cold Spring Harbor, N.Y.)).

The term “assessing” includes any form of measurement, and includes determining if an element is present or not. The terms “determining”, “measuring”, “evaluating”, “assessing” and “assaying” are used interchangeably and include quantitative and qualitative determinations. Assessing may be relative or absolute. “Assessing the presence of” includes determining the amount of something present, and/or determining whether it is present or absent. As used herein, the terms “determining,” “measuring,” and “assessing,” and “assaying” are used interchangeably and include both quantitative and qualitative determinations.

The terms “reference” and “control” are used interchangebly to refer to a known value or set of known values against which an observed value may be compared. As used herein, known means that the value represents an understood parameter, e.g., a level of expression of a cytotoxic marker gene in the absence of contact with a transfection agent.

As used herein, “treatment” or “treating” refers to inhibiting the progression of a disease or disorder, e.g., neoplastic disease or carcinoma, or delaying the onset of a disease or disorder, e.g., neoplastic disease or carcinoma, whether physically, e.g., stabilization of a discernible symptom, physiologically, e.g., stabilization of a physical parameter, or both. As used herein, the terms “treatment,” “treating,” and the like, refer to obtaining a desired pharmacologic and/or physiologic effect. The effect may be prophylactic in terms of completely or partially preventing a disease or condition, or a symptom thereof and/or may be therapeutic in terms of a partial or complete cure for a disease or disorder and/or adverse affect attributable to the disease or disorder. “Treatment,” as used herein, covers any treatment of a disease or disorder in a mammal, such as a human, and includes: decreasing the risk of death due to the disease; preventing the disease of disorder from occurring in a subject which may be predisposed to the disease but has not yet been diagnosed as having it; inhibiting the disease or disorder, i.e., arresting its development (e.g., reducing the rate of disease progression); and relieving the disease, i.e., causing regression of the disease. Therapeutic benefits of the present invention include, but are not necessarily limited to, reduction of risk of onset or severity of disease or conditions associated with neoplastic disease or carcinoma.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

### DETAILED DESCRIPTION OF THE INVENTION

Methods and compositions for determining whether a subject at least has a neoplastic disease, such as a carcinoma, are provided. In practicing the subject method, a sample from a subject is assayed for a soluble filamin analyte, such as a filamin A analyte to determine whether the subject has a neoplastic disease, such as carcinoma. Also provided are kits, systems, and devices for practicing the subject methods.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “an individual” includes one or more individuals and reference to “the method” includes reference to equivalent steps and methods known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Methods

As summarized above, the subject invention provides a method of determining whether a subject at least has a neoplastic disease. Generally the subjects are “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g., rabbits) and primates (e.g., humans, chimpanzees, and monkeys). In representative embodiments, the hosts (i.e., subjects or patients) will be humans.

The phrase “at least has a neoplastic disease” includes determining whether a subject has a neoplastic disease (i.e., making an initial diagnosis for the disease) as well determining the severity of the disease, monitoring, or staging, the progression of the disease over a course of treatment therefore, etc. In one aspect, the subject invention provides methods for determining whether a subject is suffering from a neoplastic disease. In another aspect, the subject invention provides methods for determining the severity of the neoplastic disease. In yet another aspect, the subject invention provides methods for monitoring progression of the neoplastic disease.

The phrase “neoplastic disease” is meant to include premalignant and malignant cellular growth or tumor caused by abnormal and uncontrolled cellular division. Such a disease may include a carcinoma, such as breast carcinoma and ovarian carcinoma. Such a disease may also metastasized forms localized carcinomas resulting form spreading of the cellular growth to other parts of the body through the lymphatic system or the bloodstream.

In determining whether a subject at least has a neoplastic disease, a sample from the subject is assayed to determine the presence, either qualitatively or quantiatively, of a soluble filamin analyte, e.g., in the form of the full length protein or small molecular weight fragments thereof.

In some embodiments, the filamin analyte is a soluble filamin A analyte. The amino acid sequence for filamin A is provided in GenBank Accession No. P21333, and is further described in Gorlin et al., 1990, J. Cell Biol., 111(3):1089-1105. In certain embodiments, a sample is assayed to determine the presence of the soluble form of filamin A, as well as small molecular weight fragments thereof.

In other embodiments, the filamin analyte is a soluble filamin B analyte.

The amino acid sequence for filamin B is provided in Gen Bank Accession No. 075369 and is further described in Takafuta et al., 1998, J. Biol. Chem. 273 (28), 17531-17538. In certain embodiments, a sample is assayed to determine the presence of the soluble form of filamin B, as well as small molecular weight fragments thereof.

In yet other embodiments, the filamin analyte is a soluble filamin C analyte. The amino acid sequence for filamin B is provided in Gen Bank Accession No. Q14315 and is further described in Xie et al., 1998, Biochem. Biophys. Res. Commun. 251 (3), 914-919. In certain embodiments, a sample is assayed to determine the presence of the soluble form of filamin C, as well as small molecular weight fragments thereof.

As summarized above, in practicing the subject methods a sample from a subject is assayed for the presence of a soluble filamin analyte. The sample that is assayed is a sample that is, or is derived from, any initial source that could contain a soluble filamin analyte if the subject suffers from a neoplastic disease condition. Accordingly, a suitable sample source will be derived from fluids into which the filamin analyte has been released or secreted, e.g., by neoplastic cells. The sample is generally an extracellular sample, by which is meant a sample derived from outside of cells such as extracellular biological fluid, wherein the sample does not include a cellular sample, such as, for example, a biopsy of neoplastic cells. Sample sources of interest include, but are not limited to, many different bodily fluids, e.g., serum, plasma, blood, urine, and lymph, particularly blood or blood products and urine. Sample sources of particular interest include blood samples, e.g., whole blood, serum or plasma, and urine. A sample volume of blood, serum, or urine between about 2 μl to about 2,000 μl is sufficient for determining the level of a filamin analyte. Generally, the sample volume will range from about 10 μl to about 1,750 μl, from about 20 μl to about 1,500 μl, from about 40 μl to about 1,250 μl, from about 60 μl to about 1,000 μl, from about 100 μl to about 900 μl, from about 200 μl to about 800 μl, from about 400 μl to about 600 μl.

In representative embodiments, a suitable initial source for the human sample is a blood sample. As such, the sample employed in the subject assays is generally a blood-derived sample. The blood derived sample may be derived form whole blood or a fraction thereof, e.g., serum, plasma, etc., where in some embodiments the sample is derived from blood allowed to clot and the serum separated and collected to be used to assay.

In embodiments in which the sample is a serum or serum derived sample (e.g. plasma), the sample is generally a fluid sample. Any convenient methodology for producing a fluid serum sample may be employed. In many embodiments, the method employs drawing venous blood by skin puncture (e.g., finger stick, venipuncture) into a clotting or serum separator tube, allowing the blood to clot, and centrifuging the serum away from the clotted blood. The serum is then collected and stored until assayed. Once the patient derived sample is obtained, the sample is assayed to determine the level of a filamin analyte, such as a filamin A analyte.

The subject sample may be treated in a variety of ways so as to enhance detection of a filamin analyte. For example, where the sample is blood, the red blood cells may be removed from the sample (e.g., by centrifugation) prior to assaying. Such a treatment may serve to reduce the non-specific background levels of detecting the level of a filamin analyte using an affinity reagent. Detection of a filamin analyte may also be enhanced by concentrating the sample using procedures well known in the art (e.g. acid precipitation, alcohol precipitation, salt precipitation, hydrophobic precipitation, filtration (using a filter which is capable of retaining molecules greater than 30 kD, e.g. Centrim 30™), affinity purification). In some embodiments, the pH of the test and control samples will be adjusted to, and maintained at, a pH which approximates neutrality (i.e. pH 6.5-8.0). In embodiments where the sample is urine, the pH of the sample is adjusted and the sample is concentrated in order to enhance for detection of the level of a filamin analyte.

The sample may be assayed to determine the presence and/or amount (i.e., level) of a filamin analyte using any convenient methodology. In some embodiments, the presence or amount of filamin analyte is determined by using a filamin analyte specific affinity reagent.

As reviewed above, the specific affinity reagent (e.g., a filamin A analyte specific binding reagent) is a molecule that has a high binding affinity for a filamin analyte. By high binding affinity is meant a binding affinity of at least about 10⁻⁴ M, usually at least about 10⁻⁶ M or higher, e.g., 10⁻⁹M or higher. The affinity reagent may be any of a variety of different types of molecules, so long as it exhibits the requisite binding affinity for a filamin analyte.

Any convenient assay protocol may be employed. Suitable assays that may be employed include antibody-based assays, e.g. ELISAs, such as those described in the experimental section infra. Antibody based assays require the use of antibodies, or fragments and mimetics thereof, specific for a filamin analyte. Of interest are direct assays, i.e., those which employ an antibody reagent, such as antibodies, or fragments and mimetics thereof, specific for a filamin analyte, such as filamin A analyte.

Antibodies that specifically bind to a subject filamin analyte can be prepared using a variety of convenient methods known to those of skill in the art. See Guide to Protein Purification, supra, as well as Antibodies, A Laboratory Manual (Harlow & Lane eds. Cold Spring Harbor Press, 1988).

The antibodies may be polyclonal or monoclonal antibodies depending on the nature of the intended use, as long as they are specific for a filamin analyte.

Depending on the desired use of the antibodies, in some cases monoclonal antibodies will be used, where representative antibodies include GFD.OA.p185-1, GFD-OA.p200-1 and OAB. In some embodiments, the antibody specifically binds to a soluble form of a filamin analyte such that the antibody does not bind to a non-soluble form of a filamin analyte, such as for example, the antibody specifically binds to an epitope of the soluble form of the filamin analyte that is not exposed (available for binding with an antibody) unless the filamin analyte is in the soluble form. In certain embodiments, the antibody specifically binds to a soluble form of a filamin A analyte.

For preparation of polyclonal antibodies, the first step is immunization of the host animal with a filamin analyte or an immunogenic fragment, including fragment derivatives thereof, where the filamin analyte immunogen will preferably be in substantially pure form, comprising less than about 1% contaminant. The immunogen may comprise a complete filamin analyte, fragments or derivatives thereof. To increase the immune response of the host animal, the immunogen may be combined with an adjuvant, where suitable adjuvants include alum, dextran, sulfate, large polymeric anions, oil and water emulsions, e.g. Freund's adjuvant, Freund's complete adjuvant, and the like. The immunogen may also be conjugated to synthetic carrier proteins or synthetic antigens. A variety of hosts may be immunized to produce the polyclonal antibodies. Such hosts include rabbits, guinea pigs, rodents, e.g. mice, rats, sheep, goats, and the like. The immunogen is administered to the host, usually intradermally, with an initial dosage followed by one or more, usually at least two, additional booster dosages. Following immunization, the blood from the host is collected, followed by separation of the serum or plasma from the blood cells. The Ig present in the resultant antiserum may be further fractionated using known methods, such as ammonium salt fractionation, DEAE chromatography, and the like.

As with the preparation of polyclonal antibodies, the first step in preparing monoclonal antibodies specific for a filamin analyte and fragments thereof is to immunize a suitable host, where suitable hosts include rats, hamsters, mice and the like, and are preferably mice. The filamin analyte immunogen, which as above, may be an entire filamin analyte or a fragment or derivative thereof, is administered to the host in any convenient manner, where such methods include: subcutaneous injection with adjuvants, nitrocellulose implants comprising the immunogen, intrasplenic injections, and the like, where the immunization protocol may be modulated to obtain a desired type of antibody, e.g. IgG or IgM, where such methods are known in the art. Following immunization, plasma cells are harvested from the immunized host, where sources of plasma cells include the spleen, lymph nodes and the like, with the spleen being preferred. The plasma cells are then immortalized with myeloma cells to produce hybridoma cells. A variety of myeloma cell lines are available and known to those of skill in the art. The plasma and myeloma cells are fused by combining the cells in a fusion medium usually in a ratio of about 10 plasma cells to 1 myeloma cell, where suitable fusion mediums include a fusion agent, e.g. PEG 1000, and the like. Following fusion, the fused cells are selected, e.g. by growing on HAT medium. Following hybridoma cell production, culture supernatant from individual hybridomas is screened for reactivity with a filamin analyte using standard techniques, where such screening techniques include ELISA, dot blot immunoassays and the like. The antibody may be purified from the supernatants or ascites fluid by conventional techniques, e.g. affinity chromatography with a filamin analyte bound to an insoluble support, protein A sepharose and the like.

The above prepared or obtained antibodies may be modified in a number of different ways to optimize their utility for use in a particular immunoassay. For example, antibody fragments, such as Fv, F(ab)₂ and Fab may be prepared by cleavage of the intact protein, e.g. by protease or chemical cleavage.

The antibody reagent, such as antibodies, fragments or derivatives thereof may also be labeled in order to facilitate detection. A variety of protein labeling schemes are known in the art and may be employed, the particular scheme and label chosen being the one most convenient for the intended use of the antibody, e.g. immunoassay. Examples of labels include labels that permit both the direct and indirect measurement of the presence of the antibody. Examples of labels that permit direct measurement of the antibody include radiolabels, such as ³H or ¹²⁵I, fluorescers, dyes, beads, chemilumninescers, colloidal particles, and the like. Examples of labels which permit indirect measurement of the presence of the antibody include enzymes where a substrate may provide for a colored or fluorescent product. For example, the antibodies may be labeled with a covalently bound enzyme capable of providing a detectable product signal after addition of suitable substrate. Instead of covalently binding the enzyme to the antibody, the antibody may be modified to comprise a first member of specific binding pair which specifically binds with a second member of the specific binding pair that is conjugated to the enzyme, e.g. the antibody may be covalently bound to biotin and the enzyme conjugate to streptavidin. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art.

The assay of the subject invention may be performed in solution or may use a solid (insoluble) support (e.g. polystyrene, nitrocellulose, or beads), using any standard methods (e.g., as described in Current Protocols in Immunology, Coligan et al., ed.; John Wiley & Sons, New York, 1992). Typical methods include ELISAs (enzyme-linked immunosorbent assays), IRMAs (immunoradiometric assays), and RIAs (radioimmunoassays). Where the assay is performed in solution, the test and control samples are each incubated with a filamin analyte affinity reagent for a time period sufficient to allow formation of analyte and affinity reagent complexes, preferably between about 0.1 hours up to 24 hours, or more. As previously noted, the affinity reagent may include a detectable label (e.g. radionuclide, fluorescer, or enzyme). The sample is then treated to separate the analyte and affinity reagent complexes from excess, unreacted affinity reagent (e.g. by addition of anti-affinity reagent (e.g., anti-immunoglobulin antiserum) followed by centrifugation (e.g., 1000×g for 7 min) to precipitate the analyte and affinity reagent complexes, or by binding to an affinity surface such as a second, unlabelled filamin analyte affinity reagent (e.g., antibody) fixed to a solid substrate such as Sepharose or a plastic well). Detection of affinity reagent bound to a filamin analyte may be achieved in a variety of ways well known in the art. If necessary, a substrate for the detectable label may be added to the sample.

Where the assay uses a solid support, the support will have an affinity reagent capable of specifically binding a filamin analyte, where the affinity reagent is bound to the support surface. The affinity reagent facilitates the stable, wash-resistant binding of a filamin analyte present in the sample to the solid support. The insoluble supports may be any compositions to which affinity reagents, such as antibodies or fragments and mimetics thereof can be bound, which is readily separated from soluble material, and which is otherwise compatible with the overall method of measuring a filamin analyte in the sample. The surface of such supports may be solid or porous and of any convenient shape. Examples of suitable insoluble supports to which the affinity reagent is bound include beads, e.g. magnetic beads, membranes and microtiter plates. These are typically made of glass, plastic (e.g. polystyrene), polysaccharides, nylon or nitrocellulose. Microtiter plates are especially convenient because a large number of assays can be carried out simultaneously, using small amounts of reagents and samples. Suitable affinity reagents include antibodies, or fragments and mimetics thereof, which specifically bind a filamin analyte, or anti-idiotype antibodies, or fragments and mimetics thereof, which specifically bind to the anti-filamin analyte-antibody. Alternatively, the solid support itself may bind a filamin analyte directly through the charged properties of the support surface, thus taking advantage of the highly negatively charged nature of a filamin analyte molecule. Methods for binding affinity reagents (e.g., antibodies, or fragments and mimetics thereof) to solid supports are well known in the art. After binding of the affinity reagent to the support, the support may be treated with a blocking agent, which binds to the support in areas not occupied by the affinity reagent. Suitable blocking agents include non-interfering proteins such as bovine serum albumin, casein, gelatin, and the like. Alternatively, several detergents at non-interfering concentrations, such as Tween, NP40, TX100, and the like may be used. Such blocking treatment reduces nonspecific binding.

In certain embodiments, a series of standards, containing known concentrations of filamin may be assayed in parallel with the samples or aliquots thereof to serve as controls. Generally from about 0.001 to 1 ml of sample, diluted or otherwise, is sufficient, usually about 0.01 ml sufficing. Furthermore, in certain embodiments, each sample and standard will be added to multiple wells so that mean values can be obtained for each. The test and control samples are each incubated with the solid support for a time sufficient for binding of a filamin analyte to the affinity reagent. The incubation time should be sufficient for a filamin analyte to bind the insoluble first affinity reagent. Generally, from about 0.1 hour to 3 hours is sufficient, usually 1 hour sufficing.

After incubation, the reacted samples may be washed to remove unbound or non-specifically bound material. Generally, a dilute non-ionic detergent medium at an appropriate pH, generally 7-8, is used as a wash medium. An isotonic buffer, such as phosphate-buffered saline, may be employed in the washing step. From one to six washes may be employed, with sufficient volume to thoroughly wash non-specifically bound proteins present in the sample. Preferably, the washing step will not cause dissociation of filamin analyte and affinity reagent complexes.

A second affinity reagent which specifically binds a filamin analyte is then incubated with the filamin analyte-affinity reagent complexes. In some embodiments the second affinity reagent is an anti-filamin analyte antibody, or fragment or mimetic thereof, where the second affinity reagent preferably binds to an epitope different from the epitope bound by the first affinity reagent. The second affinity reagent (e.g., antibody) used to detect a filamin analyte bound to the support may be detectably labeled to facilitate direct, or indirect detection of filamin analyte-first affinity reagent-second affinity reagent complexes. Examples of labels which permit direct measurement of immunocomplexes include radiolabels, such as ³H or ¹²⁵I, fluorescers, dyes, beads, chemilumninescers, colloidal particles, and the like. Examples of labels which permit indirect measurement of binding include enzymes where the substrate may provide for a colored or fluorescent product.

In some embodiments, the second affinity reagent (e.g., antibody or fragment and mimetic thereof) is labeled with a covalently bound enzyme capable of providing a detectable product signal after addition of suitable substrate. Examples of suitable enzymes for use in conjugates include horseradish peroxidase, alkaline phosphatase, malate dehydrogenase and the like. Where not commercially available, such affinity reagent-enzyme conjugates are readily produced by techniques known to those skilled in the art.

Alternatively, a third detectably labeled affinity reagent (e.g., antibody, or fragment and mimetic thereof) which specifically binds the second affinity reagent may be used to detect the filamin analyte-first affinity reagent-second affinity reagent complexes. Examples of third affinity reagent/second affinity reagent-specific molecule pairs include antibody/anti-antibody and avidin (or streptavidin)/biotin. Since the resultant signal is thus amplified, this technique may be advantageous where only a small amount of a filamin analyte is present in the sample. An example is the use of a labeled antibody specific to the second antibody. The volume, composition and concentration of the third affinity reagent solution provides for measurable binding to the filamin analyte already bound to the second affinity reagent. Generally, the same volume as that of the sample is used: from about 0.001 ml to about 1 ml is sufficient, usually about 0.1 ml sufficing. The concentration will generally be sufficient to saturate the filamin analyte potentially bound to second reagent.

In such assays, the concentration of the second affinity reagent may range from about 0.1 μg/ml to about 50 μg/ml, such as about 1 μg/ml. The solution containing the second antibody is generally buffered in the range of about pH 6.5 to about 9.5. The incubation time should be sufficient for the second affinity reagent to bind available molecules. Generally, from about 0.1 hour to about 3 hours is sufficient, usually 1 hour sufficing. After the second affinity reagent has bound, the insoluble support is generally again washed free of non-specifically bound second receptor, essentially as described for prior washes. After non-specifically bound material has been cleared, the signal produced by the bound conjugate is detected by conventional means. Where an enzyme conjugate is used, an appropriate enzyme substrate is provided so a detectable product is formed. More specifically, where a peroxidase is the selected enzyme conjugate, a preferred substrate combination is H₂O₂ and O-phenylenediamine which yields a colored product under appropriate reaction conditions. Appropriate substrates for other enzyme conjugates such as those disclosed above are known to those skilled in the art. Suitable reaction conditions as well as means for detecting the various useful conjugates or their products are also known to those skilled in the art. For the product of the substrate O-phenylenediamine for example, light absorbance at 490-495 nm is conveniently measured with a spectrophotometer.

Alternatively, a filamin analyte, such as filamin A analyte, may be detected by using a competitive binding assay. The test and control samples are incubated with the anti-filamin affinity reagent as described above, to allow for formation of filamin analyte-affinity reagent complexes. The affinity reagent may be fixed to a solid surface or in solution. After washing to remove unbound material from the precipitated filamin analyte-affinity reagent complexes or from the solid support (if any) to which the antibody is fixed, the samples are then incubated with a standard amount of detectably labeled filamin, detectably labeled recombinant hybrid filamin, or a detectably labeled fragment of filamin which retains the ability to compete with a native filamin analyte for binding sites on the anti-filamin analyte binding reagent. Binding is detected by standard means: e.g., by measuring the amount of label associated with (a) the solid support (if any), or (b) the precipitated analyte/binding agent complexes. A lower level of binding of the detectably labeled filamin in the test sample than in the negative control indicates the presence of an elevated level of filamin analyte in the test sample.

Alternatively, the binding of the second filamin molecule used in the competitive binding assay (i.e. the filamin introduced into the test sample after incubation of the test sample with the anti-filamin analyte affinity reagent), may be measured by means of an epitope present on the second filamin molecule which is absent in a filamin analyte derived from a sample of bodily fluid. For example, the second filamin molecule may be a recombinant fusion protein which retains the ability to bind competitively to the affinity reagent used in the assay. Binding of filamin fusion protein to the anti-filamin affinity reagent may then be detected by incubating the sample with a detectably labeled second affinity reagent which specifically binds the fusion protein and does not bind the filamin analyte from the sample. An example of a recombinant filamin fusion protein is one that containing an N-terminal extension of amino acids, which recombinant filamin fusion protein may be used in such a detection method, since affinity reagents which specifically bind to the N-terminal amino acid extension of the recombinant molecule would not be expected to bind to a filamin analyte present in a sample. Examples of other epitopes which may be introduced into a filamin fusion protein include epitopes for use as targets for chemical modification and epitopes which have an altered amino acid sequence relative to a naturally-occurring filamin analyte (to provide a peptide epitope absent in a filamin analyte).

As summarized above, the subject methods are used to determine at least the presence and often the amount (i.e., level) of a filamin analyte, such as a filamin A analyte, present in sample from a subject to determine whether the subject at least has a neoplastic disease, such as breast carcinoma, ovarian carcinoma, brain cancer, prostate cancer, gastric cancer, and the like. Specifically, in some embodiments, determining the level of a filamin analyte in a sample according to the subject methods includes a step of comparing the detected signal obtained from the subject methods to a table or other source of predetermined values or reference values (collectively referred to herein as a reference) which provide information about the disease activity in the host, e.g., that positively or negatively correlate to the presence of the neoplastic disease involving abnormal levels of a filamin analyte, a particular stage of the disease involving abnormal levels of a filamin analyte, and the like. For example, a table of values may be consulted in this step, such as a reference, where the table comprises representative values for a filamin analyte as found in patients having at least neoplastic disease (e.g., breast carcinoma, or ovarian carcinoma) involving abnormal levels of a filamin analyte. The values may be presented in numerical form, in picture form (e.g. as bands on a gel), and the like. By comparing the observed values with these reference values, e.g., by comparing a pattern of a filamin analyte in the sample to a reference pattern or picture, characterization of the disease activity, e.g., confirmation of diagnosis, determination of disease state, responsiveness to treatment or therapy, and the like, is readily made.

In other embodiments, determining the level of a filamin analyte in a sample according to the subject methods involves comparing the level of anti-filamin affinity reagent binding in the test sample to the level of anti-filamin affinity reagent binding in the negative and/or positive control samples. Appropriate control samples for the assay include blood, serum, or urine collected from subjects who do not have a neoplastic disease (i.e., a negative control), or samples which contain a known, predetermined amount of a filamin analyte (i.e., a positive control).

In such embodiments the level of affinity reagent binding in the test sample is compared to a range of negative and positive control sample, in which the positive control samples have a range of predetermined quantities of filamin present, and the negative control samples do not have any filamin present.

Utility

The subject methods may be used to determine whether a subject at least has a neoplastic disease. As indicated above, the invention provides methods for assaying a sample from a subject to determine the presence, and often amount or level of a filamin analyte present in the sample. The phrase “at least has” is used broadly to refer to any type of information about the state of the neoplastic disease involving abnormal levels or amounts of a filamin analyte present in a sample from the subject, such as the soluble form of filamin A, or fragment thereof. As such, the subject methods may be used to facilitate diagnosis of a neoplastic disorder prior to or coincident with the onset of clinical symptoms, confirm an initial diagnosis of a neoplastic disease involving abnormal levels of a filamin analyte, to determine the state (i.e., severity) of the neoplastic disease in a patient known to have a neoplastic disease involving abnormal levels of a filamin analyte, to monitor the progression, for example staging, of the neoplastic disease, responsiveness of the neoplastic disease to treatment or therapy, and the like.

Where the subject method is employed to confirm an initial diagnosis of a neoplastic disease (e.g., carcinoma, including breast carcinoma and ovarian carcinoma, brain cancer, prostate cancer, gastric cancer, and the like), a sample is obtained from a subject. In such embodiments, the subject may be identified as presenting the classical symptoms of neoplastic disease, or have a medical history that indicates susceptibility to neoplastic disease. The sample is assayed for the level of a filamin analyte present, and then compared to reference values, where the reference values correlate a detected level amount with a neoplastic disease. In some embodiments, the sample is assayed for the level of a filamin A analyte present.

Classical symptoms for breast carcinoma including, for example, a lump or thickening in the breast or underarm, change in size or shape of the breast, nipple discharge or nipple turning inversion, redness or scaling of the breast skin or nipple, and ridges or pitting of the breast skin. Traditionally, once a patient presents with symptoms suggestive of a breast carcinoma or an abnormal screening mammogram, such patients will generally be referred for further diagnostic mammograms of, for example, the suspicious areas of the breast, or an ultrasound. Depending on the results of the mammograms and/or ultrasounds, a physician or clinician may recommend that a biopsy of the breast tissue be taken. Once tissue is removed, and reviewed by a pathologist a diagnosis will be made as whether the sample is cancerous or is not cancerous. If cancerous, the neoplastic growth will be characterized based on factors well known in the act, such as, for example, the type of tissue, the invasiveness of the growth, the grade, and the like.

In order to guide treatment and offer some insight into prognosis, breast cancer is staged into five different groups. The stages of breast carcinoma are as follows: Stage 0, also referred to as carcinoma in situ, includes Lobular carcinoma in situ (LCIS), which refers to abnormal cells lining a gland in the breast, and Ductal carcinoma in situ (DCIS), which refers to abnormal cells lining a duct. Women with DCIS have an increased risk of getting invasive breast cancer in that breast; Stage I, wherein the breast carcinoma is less that 2 cm across and has not spread beyond the breast, Stage II, wherein the breast carcinoma is either less than about 2 cm across and has spread to the lymph nodes under the arm; or the carcinoma is between about 2 cm and about 5 cm (with or without spreading to the lymph nodes under the arm); or the carcinoma is greater than about 5 cm and has not spread outside the breast; Stage III, wherein the breast carcinoma is locally advanced and the carcinoma is greater than 5 cm across and has spread to the lymph nodes under the arm, or the carcinoma is extensive in the underarm lymph nodes, or the carcinoma has spread to lymph nodes near the breastbone or to other tissues near the breast; and Stage IV, wherein the breast carcinoma has spread (metastasized) to a distant location, such as to other organs in the body.

While ovarian carcinoma does not present with classical symptoms, particularly in the early stages of the disease, ovarian carcinoma does however present a variety of problems including, for example, abdominal swelling or abdominal pain, vaginal bleeding between periods or after menopause, bloating, gas, indigestion or cramps, pelvic pain, loss of appetite, feeling full after a small meal, or feeling full very easily, changes in bowel or bladder habits, and weight loss or weight gain. Traditionally ovarian carcinoma has been diagnosed by a physician during a pelvic examination and followed by surgery and treatment. In order to guide treatment and offer some insight into prognosis, ovarian cancer is staged into four different groups at the time of the surgery. The staging system for ovarian carcinoma includes the following: Stage I, ovarian carcinoma is confined to the ovary or ovaries; Stage II, ovarian carcinoma has spread beyond the ovaries, but is confined to the pelvis (e.g., uterus, bladder or rectum); Stage III, ovarian carcinoma has spread to the peritoneum (i.e., the lining of the abdomen) and/or lymph nodes; and Stage IV, ovarian carcinoma has spread (metastasis) to a distant location, for example to other organs. Generally, the higher the stage, the more serious the cancer.

Advanced prostate cancers can cause a variety of symptoms including: trouble starting urination, urinating much more frequently than usual, the feeling that all of the urine can't be released, pain upon urination or ejaculation, blood in the urine or semen, impotence, bone pain, and the like. However, most early prostate cancers are detected by a physician using a digital rectal exam or by using a prostate specific antigen (PSA) assay before any symptoms arise. Prostate cancer is divided into four different stages to help guide treatments and offer information about the chances for a cure. The staging system includes the following: Stage I: tumor cannot be felt during a digital rectal exam; it was detected by an elevated PSA blood test or incidentally found during another prostate procedure for a benign condition; Stage II: tumor can be felt during a digital rectal exam, but it has not spread beyond the prostate and it hasn't spread to lymph nodes or other organs; Stage III: tumor extends outside the prostate and can be in the seminal vesicles, but not in any other organs or lymph nodes; and Stage IV: tumor has spread to other organs or lymph nodes.

Symptoms of gastric cancer are often nonspecific. The vast majority of gastric cancer patients present with vague complaints such as upper abdominal discomfort or indigestion, loss of appetite, occasional vomiting, belching, or decreased ability to eat a large meal. Unfortunately, these symptoms are often the exact symptoms that patients experience when they have peptic ulcer disease or gastritis. Therefore, patients can be treated for benign diseases, such as ulcers, without the diagnosis of gastric cancers being made. In order to guide diagnosis and treatment, gastric cancer can be staged as follows using the Modified Astler-Coller system: Stage A: lymph nodes not involved; tumor very superficial in stomach; Stage B1: lymph nodes not involved; tumor deeper, but still within stomach wall (not entire way through); Stage B2: lymph nodes not involved; tumor through the stomach wall: Stage B3: lymph odes not involved; tumor invading into other organs: Stage C1: lymph nodes involved, but tumor not through the stomach wall; Stage C2: lymph nodes involved and tumor through the stomach wall: and Stage C3: lymph nodes involved and tumor invading into other organs.

The symptoms of both primary and metastatic brain tumors depend mainly on the location in the brain and the size of the tumor. Since each area of the brain is responsible for specific functions, the symptoms will vary a great deal. Tumors in the frontal lobe of the brain may cause weakness and inability to move on one side of the body, known as paralysis, mood disturbances, difficulty thinking, confusion and disorientation, and wide emotional mood swings. Parietal lobe tumors may cause seizures, numbness or paralysis, difficulty with handwriting, inability to perform simple mathematical problems, difficulty with certain movements, and loss of the sense of touch. Tumors in the occipital lobe can cause loss of vision in half of each visual field, visual hallucinations, and seizures. Temporal lobe tumors can cause seizures, perceptual and spatial disturbances, and inability to understand simple of multi-step commands, known as receptive aphasia. If a tumor occurs in the cerebellum, the person may have difficulty maintaining their balance, known as ataxia, loss of coordination, headaches, and vomiting. Tumors in the hypothalamus may cause emotional changes, and changes in the perception of hot and cold. In addition, hypothalamic tumors may affect growth and nutrition in children. With the exception of the cerebellum, a tumor on one side of the brain causes symptoms and impairment on the opposite side of the body. For example, a tumor on the left side of the brain may cause numbness in the right arm.

The subject methods are also employed to determine the stage (i.e., severity) of the neoplastic disease in a subject already known to have a neoplastic disease, such as breast carcinoma or ovarian carcinoma. In other words, the subject method can be used to determine whether the subject suffering from a neoplastic disease involving an abnormal level of a filamin analyte is in a remission stage, a chronic stage etc. For example, the subject methods may be employed to determine a clinical remission of breast carcinoma or ovarian carcinoma. To determine the stage of the disease, the observed values (e.g., level) for a filamin analyte in the assayed sample are compared to reference values that are correlated to a particular stage of disease involving abnormal levels a filamin analyte.

In some embodiments, the severity of a neoplastic disease may be determined by quantitating the amount of a filamin analyte in the test sample, or by determining the relative amount compared to standard controls or a reference. For example, quantitation of a filamin analyte may be achieved by comparing the level of affinity reagent (e.g., antibody, or fragment or mimetic thereof) binding in the test sample to the level of affinity reagent binding in one or more identically treated control samples containing known amounts of a filamin analyte, or by comparing the test sample signal to a table of standard values (e.g., reference). Where a competitive binding assay is employed, the levels of binding of detectably labeled affinity reagent to a filamin analyte in a sample from a subject may be correlated with the levels of binding of the detectably labeled affinity reagent in control samples having a known amount of filamin. The level of a filamin analyte present in the test sample may then be correlated with a degree of disease severity and patient prognosis by reference to these controls. Quantitation of a filamin analyte in the test sample may alternatively be achieved by precipitation of the filamin analyte-affinity reagent complexes from solution and comparison of the level of protein in the test sample precipitate relative to precipitates of control samples having a known amount of a filamin analyte.

In yet other embodiments, characterization of disease activity yields information concerning progression of the neoplastic disease in the subject, e.g. whether progression of the neoplastic disease has accelerated or slowed. For example, the initial characterization date, i.e., the level of a filamin analyte present in the sample derived from the subject, could be employed as a baseline value to evaluate subsequent testings, e.g. at some time following the initial testing, e.g. 3 months. If the level of a filamin analyte decreases in subsequent testing, this indicates that the neoplastic disease is not progressing and may be resolving. Alternatively, if the level of a filamin analyte increases, this indicates that the neoplastic disease is progressing in severity.

In some embodiments, the subject methods of the present invention may be used in the treatment a subject for a neoplastic disease. In such embodiments, the subject methods are employed to first determine whether a subject suffers from a neoplastic disease (or the severity of the disease) by determining the level of a filamin analyte in a sample derived from the subject according to the subject methods. Once, a determination has been made with respect to whether the subject suffers from a neoplastic disease, a treatment protocol is identified for the subject based on the determination of the level of a filamin analyte in a sample derived from the subject.

Treatment protocols for neoplastic disease are well know in the art and include, but are not limited to, surgery, chemotherapy, radiotherapy, and the like. Treatment protocols for breast carcinoma are well known in the art and include, but are not limited to, surgery, radiotherapy chemotherapy, including treatment regimens such as AC (doxorubicin and cycolphosphamide) for and CMF (cyclophosphamide, methotrexate, and fluorouracil), hormonal therapy, such as Tamoxifen, biological therapy, such as Herceptin, and the like. Treatment protocols for ovarian carcinoma are well known in the art and include, but are not limited to, surgery, radiotherapy chemotherapy, including, treatment with Paclitaxel plus either Cisplatin or Carboplatin (platinum containing drugs), treatment with other compounds, such as Gemcitabine and Doxorubicin.

In other embodiments, characterization data of the level of a filamin analyte present in a sample derived from a subject obtained by the subject methods may also be used to determine whether a particular therapeutic regimen is having positive affects with respect to the progression of the neoplastic disease, such as breast carcinoma and ovarian carcinoma. For example, at various time periods during the course of treatment, the subject methods may be performed to obtain a reading of the amount of a filamin analyte present in a sample derived from a subject under a particular treatment regimen. If the level of a filamin analyte is increasing, this indicates that the treatment regimen is not having the desired effect, where the desired effect is to slow the progression of the neoplastic disease. Alternatively, if the level of a filamin analyte is decreasing, this indicates that the treatment regimen is working with respect to slowing the progression of the neoplastic disease.

Kits

Also provided are kits that find use in practicing the subject methods, as described above. The kits for practicing the subject methods at least include reagents for assaying a sample for a filamin analyte, where such kits may include: filamin analyte specific affinity reagents, such as an antibody, or fragment or mimetic thereof, and/or immunoassay devices comprising the same members of a signal producing system, such as antibodies, enzyme substrates, and the like; various buffers for use in carrying out the subject detection assays; a reference for determining the amount of a filamin analyte in a sample; and the like. In some embodiments, the filamin analyte is a filamin A analyte.

The kits may further include one or more reagents necessary for preparation of the patient derived sample, such as heparin, Ficoll-Hypaque, lysing buffer, protease inhibitor, and the like, e.g. where the patient sample is PBMC derived, etc. In addition, the subject kits may further include one or more components employed in fractionation of the sample, such as an electrophoretic medium or precursors thereof, e.g. dried precursors of polyacrylamide gels, one or more buffer mediums or components thereof, and the like.

In certain embodiments, the kits further include at least an information storage and presentation medium that contains reference data with which assay results may be compared in order to diagnose and/or characterize the neoplastic disease involving abnormal levels of a filamin analyte in the subject being assayed, i.e., reference data that that positively or negatively correlate to the presence of the neoplastic disease involving abnormal levels of a filamin analyte, a particular stage of the disease involving abnormal levels of a filamin analyte, and the like. The information storage and presentation medium may be in any convenient form, such as a printed information on a package insert, an electronic file present on an electronic storage medium, e.g. a magnetic disk, CD-ROM, and the like. In yet other embodiments, the kits may include alternative means for obtaining reference data, e.g. a website for obtaining the reference data “on-line.”

The kits may further include means for obtaining the patient sample, e.g., a syringe. The subject kits further typically include instructions for carrying out the subject methods, where these instructions may be present on a package insert and/or the packaging of the kit. Finally, the kits may further include one or more reagents from an additional biochemical assay which is used to detect the presence of and/or characterize the neoplastic disease involving abnormal levels a filamin analyte.

The kit components may be present in separate containers, or one or more of the components may be present in the same container, where the containers may be storage containers and/or containers that are employed during the assay for which the kit is designed.

Systems

Also provided are systems that find use in practicing the subject methods, as described above. The systems for practicing the subject methods at least include reagents for assaying a sample for a filamin analyte, where such systems may include: filamin analyte affinity reagents, such as an antibody, or fragment or mimetic thereof, and/or immunoassay devices comprising the same members of a signal producing system, such as antibodies, enzyme substrates, and the like; various buffers for use in carrying out the subject detection assays; a reference for determining the amount of a filamin analyte in a sample; and the like. In some embodiments, the filamin analyte is a filamin A analyte.

Furthermore, additional items that are required or desired in the protocol to be practiced with the system components may be present, which additional items include, but are not limited to: means for obtaining the patient sample, e.g. a syringe; one or more reagents necessary for preparation of the patient derived sample, such as heparin, Ficoll-Hypaque, lysing buffer, protease inhibitor, and the like; instructions for carrying out the subject methods; one or more reagents from an additional biochemical assay which is used to detect the presence of and/or characterize the neoplastic disease involving abnormal levels a filamin analyte.

Devices

Also provided are devices that find use in practicing the subject methods, as described above. Devices for practicing the subject methods at least include reagents for assaying a sample derived from a subject for a filamin analyte, where such devices may include: filamin analyte specific affinity reagents, such as an antibody, or fragment or mimetic thereof, immobilized on the surface of a solid support. In some embodiments, the filamin analyte is a filamin A analyte.

Additional items that are required or desired in the methods to be practiced with the devices may be present, which additional items include, but are not limited to: means for obtaining the patient sample, e.g. a syringe; one or more reagents necessary for preparation of the patient derived sample, such as heparin, Ficoll-Hypaque, lysing buffer, protease inhibitor, and the like; instructions for carrying out the subject methods using the subject devices; one or more reagents from an additional biochemical assay which is used to detect the presence of and/or characterize the neoplastic disease involving abnormal levels a filamin analyte.

A number of such devices are known in the art. In one non-limiting example, the apparatus will generally employ a continuous flow-path of a suitable filter or membrane, having at least three regions, a fluid transport region, a sample region, and a measuring region. The sample region is prevented from fluid transfer contact with the other portions of the flow path prior to receiving the sample. After the sample region receives the sample, it is brought into fluid transfer relationship with the other regions, and the fluid transfer region contacted with fluid to permit a reagent solution to pass through the sample region and into the measuring region. The measuring region may have bound to it the first affinity reagent, and second labeled affinity reagent combined with the assayed sample and the sandwich assay performed as above.

In another non-limiting example the device is a dipstick, to the surface of which is bound an affinity reagent, such an antibody, or fragment or mimetic thereof, which specifically binds a filamin analyte. In such an exemplary device, the dipstick is inserted directly into a test sample (e.g., blood, serum, or urine) derived from a subject for a period of time sufficient to permit binding of a filamin analyte to the affinity reagent bound to the dipstick. The dipstick may be then withdrawn and, if necessary, washed to remove nonspecifically bound material. The dipstick is then inserted into a container containing a detectably labeled second affinity reagent, such an antibody, or fragment or mimetic thereof, which specifically binds a filamin analyte. After incubation for a time sufficient for binding of the second antibody to the filamin analyte-affinity reagent complexes, the dipstick may be washed and binding of the second affinity reagent detected by standard means. Where necessary for detection of the second antibody, the dipstick may be inserted into a second container containing a reagent which activates the detectable label on the second antibody.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

The following materials and methods are used in the examples below.

Electron Microscopy

MDA.MB.231 cells were washed several times with PBS and then fixed with 2.5% Glutaraldehyde or 10% Formalin and incubated at 4° C. and room temperature for 2 hour, respectively. Cells were scraped and after centrifugation the cell pellets were further processed for gold and conventional EM studies.

Immunoprecipitation and Western Blot Analysis

Cells (1×10⁶ cells) in tissue culture were lysed in RIPA buffer; 250 mM NaCl, 20 mM Na₂HPO₄, 1% Triton, 1% deoxycholic acid, 0.1% sodium dodecyl sulfate, protease inhibitor (Complete Mini, Roche), 1 mM NaVO₄.

Total protein (800 μg/ml) was immunoprecipitated with the following mouse monoclonal antibody specific to phosphotyrosine (4G10, Upstate, Lake Placid, N.Y.), resolved by 4-12% SDS-PAGE gels and transferred to nitrocellulose sheets. Blotting was performed with anti-integrin alpha-6 antibody (GoH3, NeoMarkers, Fremont, Calif.), anti-p200 antibody and anti-actin MoAb (119, Santa Cruz Biotechnology, Calif.). Bands were visualized with secondary HRP-conjugated antibodies and the ECL system (Amersham Pharmacia). The blots were scanned by use of Chemilmager™ 5500 (Alpha Innotech Corporation, San Leandro, Calif.).

Matrigel Outgrowth and Indirect Immunofluorescent Staining

Cells were seeded on glass coverslips in 12-well plates coated with 0.5 ml of Matrigel (10 mg/ml; kindly provided by Dr. Hynda Kleinman, National Institute of Dental and Craniofacial Research, Bethesda, Md.). The plates were incubated at 37° C. in IMEM medium (Gibco) containing 10% FBS for various time periods. After the nonadherent cells were removed by washing gently with PBS, adherent cells were fixed with 70% methanol for 5 minutes at room temperature and stained with Giemsa (Sigma, St. Louis, Mo.). The images were analyzed using an Olympus IX-70 Laser Confocal Scanning Microscope equipped with an Olympus 60×/1.4 N.A. objective lens.

Amino Acid Analysis of p280

The proteins on the western blotted membranes were stained with Ponceau S. Individual protein bands were excised and washed with pure water. The protein samples on PVDF were reduced and digested with Achromobacter protease I (Wako) in situ as described (Iwamatsu, et al., 1992 Electrophoresis, 13(3):142-7). Peptides were extracted with 5% caetonitrile and separated by HPLC on a Vydac C18 reverse-phase column using a 0-95% acetonitrile gradient in 0.1% trifluoroacetic acid. Individual peaks were dried, suspended in water and applied for gas-phase sequencing. Amino acid sequences of the amino-terminal portion of the purified fragments were dtermined by a protein sequencer (Model 477A, Applied Biosystems Inc.) equipped with an on-line PTH-analyzer (Model 120A).

Cell Lines

A human epidermoid vulva carcinoma cell line A-431; human stomach carcinoma cell lines KATO-3, MKN-28, MKN-45, MKN-74, OKAJIMA; human pancreatic carcinoma cell lines ASPC-1, PSN-1; human breast carcinoma cell lines SK-BR-3, MCF-7, MDA-MB-231, MDA-MB-361, ZR-75-1, BT-20, BT-474, BT-549; human normal breast cell lines HMEC, MCF-10A and human normal fibroblasts FS-4 were provided from the Japanese Cancer Research Resources (Tokyo, Japan) and ATCC. The cancer cell lines were cultured either in RPMI-1640 supplemented with 5% heat-inactivated FCS (Boehringer-Mannheim, Germany) or modified-IMEM supplemented with 10% FBS (Gibco-Invitrogen, NY, USA), penicillin (100 units/ml) and streptomycin (200 mg/ml). Human normal cell lines HMEC and MCF-10A were cultured in MEGM supplemented with BPE, hEGF, Insulin, Hydrocortisone, GA (Clonetics, USA) and DMEM/F12 (1:1 mixture) supplemented with 5% horse serum, penicillin/streptomycin (50 μg/ml), insulin (10 μg/ml), hydrocortisone (500 ng/ml) and EGF (20 ng/ml) respectively. Mouse myeloma cell line p3/X63-Ag8U1 kindly provided by Pathology Division, National Cancer Research Institute (Tokyo, Japan) was cultured in RPMI-1640 medium with 10% heat-inactivated FCS (Hyclone Laboratories Inc., Logan, Utah) in a humidified atmosphere of 5% CO₂/95% air at 37° C.

Preparation of Culture Medium for Immunization

A-431 and KATO-3 human vulva and stomach carcinoma cells were seeded into culture flasks containing 50 ml of RPMI-1640 with 5% FCS. When cells reached confluency, they were washed with serum-free medium and then 50 ml of serum-free media was added and the cells were incubated for 72 hours at 37° C. The media was collected aseptically and freed from cell debris. A total of 200 ml of serum-free media obtained was concentrated 20-80 fold by ultracentrifugation under nitrogen using a Diaflo cell type 8200 (Amicon Corp., Danvers, Ma.) fitted with a Diaflo YM-10 membrane (nominal M r, cutoff, 10,000; Amicon). The concentrates were sterilized with 0.22 μM Millipore filter unit and stored at −80° C.

### Example 1

### Characterization of P280 (Filamin A)

An amino acid sequence analysis was performed on the amino-terminal portions of the purified fragments that were detected by western blot ananlsys. As demonstrated by Table 1, the amino acid sequence analysis by mass spectrophotometry confirmed that the polypeptide that was identified by the p280 monoclonal antibody was filamin A. As shown in Table 1, all the fragments have a 100% homology with the amino acid sequence of filamin A, as provided in GenBank Accession No. P21333. Accordingly, these results show that the detected polypeptide is filamin A.

TABLE 1
Sequencing of p280 (filamin A) by Mass Spectrophotometry

| | Total | | |
|---|---|---|---|
| Mass | Score | Description | DB |
| 280585.9 | 1080 | (P21333) Filamin A (Alpha-filamin) | SwissProt |
| | | (Filamin 1) (Endothelial | human |

| Precursor | Ion | | Start |
|---|---|---|---|
| Mass | Score | Peptide | AA |
| 767.5519 | 59.68 | AEAGVPAEFSIWTR | 2251 |
| 715.5242 | 52.45 | AFGPGLQGGSAGSPAR | 1072 |
| 613.4237 | 9.51 | CAPGVVGPAEADIDFDIIRNDNDTFTVK | 810 |
| 576.4474 | 40.54 | DKGEYTLVVK | 2622 |
| 792.1157 | 3.57 | EAGAGGLAIAVEGPSKAEISFEDR | 2265 |
| 613.4276 | 56.6 | EATTEFSVDAR | 1273 |
| 832.0647 | 9.63 | FIPRENGVYLIDVK | 2392 |
| 573.4511 | 69.75 | GAGSGELKVTVK | 509 |
| 786.0735 | 58.96 | GAGTGGLGLAVEGPSEAK | 1382 |
| 613.4237 | 6.53 | GAGTGGLGLAVEGPSEAKMSCMDNK | 1382 |
| 542.4171 | 8.16 | GLQPK | 489 |
| 550.4224 | 50.18 | GTVEPQLEAR | 428 |
| 708.5218 | 62.8 | IANLQTDLSDGLR | 64 |
| 486.4074 | 28.42 | LLGWIQNK | 172 |
| 643.516 | 49.54 | LPQLPITNFSR | 180 |
| 533.3987 | 19.24 | LSPFMADIR | 656 |
| 767.59 | 62.11 | SPFSVAVSPSLDLSK | 959 |
| 549.7631 | 63.47 | TGVAVNKPAEFTVDAK | 685 |
| 567.1279 | 65.46 | TGVELGKPTHFTVNAK | 885 |
| 544.9062 | 39.49 | TPCEEILVK | 2599 |
| 882.5983 | 44.9 | VANPSGNLTETYVQDR | 1297 |
| 908.1664 | 92.85 | VAQPTITDNKDGTVTVR | 1815 |
| 827.114 | 42.65 | VTAQGPGLEPSGNIANK | 384 |
| 642.5321 | 53.94 | VTVLFAGQHIAK | 356 |
| 700.9771 | 42.13 | YGGDEIPFSPYR | 1622 |
| 645.9818 | 7.15 | YGGQPVPNFPSK | 1235 |

### Example 2

### Filamin A is Present in Normal Breast Cells and Breast Cancer Cells

Samples of normal, that is non transformed, breast cells as well as breast cancer cells were examined to determine whether filamin A is present. Expression of p280 (filamin A) was detected in cell lysates of MCF-10A and human normal epithelial cells (HMEC) using p280 and GoH3 monoclonal antibodies by Western blot analysis.

In addition, expression of filamin A was also confirmed in human breast cancer cell lines, including MCF-7, SK-BR-3, and MDA-MB-231. Lysates of the cell lines were assayed using p280 monoclonal antibodies by Western blot analysis. These results showed that normal breast cells have high levels of filamin A expression while breast cancer cells have a decreased level of expression of filamin A, as compared to normal breast cells.

### Example 3

### Filamin A is Secreted by Breast and Ovarian Carcinoma Cells

Breast and ovarian carcionoma cell lines were then assayed to determine whether the soluble form of filamin A is secreted. The presence of soluble filamin was detected in the culture medium of human breast carcinoma cell lines MCF-7, SK-BR-3, and MDA-MB-231 by using the filamin A specific monoclonal antibody in western blot analysis. However, the presence of soluble filamin A in culture medium of normal human breast cells MCF-10A was not observed by using the filamin A specific monoclonal antibody in western blot analysis. These results clearly indicate that filamin A is secreted by various breast cancer, but not normal cells.

Moreover, the presence of filamin A was also confirmed when large volumes of culture medium conditioned by MDA-MB-231 cells were immunoprecipitated and blotted with filamin A specific monoclonal antibody. The presence of soluble filamin A was also detected in culture medium conditioned by various human ovarian carcinoma cells including OVCAR-3, OVCAR-4, OVCAR-8, OVCA-420, OVCA-429, OVCA-432 and OVCA-433 when blotted with filamin A specific monoclonal antibody. These results show that filamin A is secreted by neoplastic cells but not normal cells.

### Example 4

### Cellular Localization of Filamin A

The expression of filamin was analyzed in human breast normal and carcinoma cells by laser confocal microscopy when seeded and cultured on Matrigel. Indirect immunofluorescent staining was observed in vesicular structures of MDA-MB-231 breast carcinoma cells while normal breast cells did not show any vesicular but a membranous staining with p280 moab and cy-5 phalloidin for cytoskeleton. Similar vesicular staining pattern was obtained for other breast carcinoma cell lines including MCF-7, SK-BR-3, BT20, BT474, BT549, ZR-75-1, and MDA.MB.361 using p280 moab.

Localization of filamin A in breast carcinoma cell lines was examined to determine if filamin A secreted by the cells. The expression and co-localization of filamin was detected using a filamin A specific monoclonal antibody (p280 moab), and lysotracker in lysosomes of MDA-MB-231, MCF-7, ZR-75-1 breast cancer cell lines when analyzed by laser scanning confocal microscopy. The results showed that filamin A is present in lysosomes in the breast carcinoma cell lines. These results also showed that filamin A is present in secretory lysosomes confirming that the carcinoma cell is secreting filamin A.

The breast carcinoma cell line MDA.MB.231 was analyzed for filamin A expression by staining with a filamin A specific monoclonal antibody (p280 moab). The immunogold labeling showed the staining of huge vesicles in MDA.MB.231 cells. These results show that filamin A is present in secretion vesicles confirming that the carcinoma cell is secreting filamin A.

It is evident from the above results that the subject invention provides an exceptionally accurate and easy to perform assay for diagnosing and monitoring or characterizing neoplastic disease conditions in a host. As such, the subject invention represents a significant contribution to the art.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of diagnosing whether a subject has a neoplastic disease, said method comprising:
assaying an extracellular sample from said subject for a soluble filamin analyte to diagnose whether said subject has said neoplastic disease.

2. The method according to claim 1, wherein said soluble filamin analyte is a soluble filamin A analyte.

3. The method according to claim 1, wherein said extracellular sample is qualititatively assayed for said soluble filamin analyte.

4. The method according to claim 1, wherein said extracellular sample is quantitatively assayed for said soluble filiman analyte.

5. The method according to claim 1, wherein said assaying employs a filamin analyte affinity reagent.

6. The method according to claim 5, wherein said filamin analyte affinity reagent is an antibody reagent.

7. The method according to claim 5, wherein said assaying employs two distinct filamin analyte affinity reagents.

8. The method according to claim 1, wherein said method is a method of monitoring progression of said neoplastic disease of said subject.

9. The method according to claim 1, wherein said extracellular sample is a blood sample.

10. The method according to claim 1, wherein said neoplastic disease is a carcinoma.

11. The method according to claim 1, wherein said carcinoma is breast cancer.

12. The method according to claim 1, wherein said carcinoma is ovarian cancer.

13. A method of treating a subject having a neoplastic disease, said method comprising:
(a) diagnosing whether said subject suffers from a neoplastic disease by assaying an extracellular sample from said subject for a soluble filamin analyte; and
(b) treating said subject based on results from said diagnosing step (a).

14. The method according to claim 13, wherein said soluble filamin analyte is a soluble filamin A analyte.

15. A kit for use in diagnosing whether a subject has a neoplastic disease, comprising:
reagents for assaying a sample for a soluble filamin analyte; and
a reference.

16.-28. (canceled)

29. A device for use in diagnosing whether a subject suffers from a neoplastic disease, said device comprising:
a soluble filamin analyte specific affinity reagent immobilized on a surface of a solid support.

30. The device according to claim 29, wherein said soluble filamin analyte is a soluble filamin A analyte.

31. The device according to claim 29, wherein said soluble filamin analyte specific affinity reagent comprises an antibody or binding fragment thereof.

32. The device according to claim 29, wherein said antibody is a monoclonal antibody.

33. An antibody that specifically binds to a soluble form of a filamin analyte.

34-38. (canceled)
